(19) United States
(12) Patent Application Publication
(10) Pub. No.: US 2014/0324132 A1
(43) Pub. Date: Oct. 30, 2014

(54) Therapeutic Device Using Far-infrared Radiation

(71) Applicant: Albert Chin-Tang Wey, Westmont, IL (US)

(72) Inventor: Albert Chin-Tang Wey, Westmont, IL (US)

(21) Appl. No.: 13/870,773

(22) Filed: Apr. 25, 2013

(51) Int. Cl. A61N 5/06

(52) U.S. Cl. CPC A61N 5/06; A61N 2005/0656; A61N 2005/0659
USPC 607/88

(57) ABSTRACT

This invention relates to a therapeutic device for treating human or animal body, comprising an array of infrared-emitting elements in an attachment means that follows the contour of the body part to be treated, said infrared-emitting element being an infrared-emitting ceramic plate made of a mixture of infrared-emitting oxides having specific spectral luminance covering at least a part of 3-20 um (micrometer) wavelength spectrum, that provides an effective means to healing the body. A locally administrable heating means may be used for escalated healing effects.

### BACKGROUND

1. Field of Invention

This invention relates to a therapeutic device for treating human or animal body, comprising an array of infrared-emitting elements in an attachment means that follows the contour of the body part to be treated, said infrared-emitting element being an infrared-emitting ceramic plate made of a mixture of infrared-emitting oxides having specific spectral luminance covering at least a part of 3-20 um (micrometer) wavelength spectrum, that provides an effective means to healing the body. A locally administrable heating means may be used for escalated healing effects.

2. Description of Prior Art

According to Organic Chemistry photoexciting molecules of water and organic compounds with infrared (IR) photons shorter than 20 um (micrometer) in wavelength can increase their vibrational energy, in forms of stretching and/or bending vibrations. Such effects have been described in textbooks. The photoexcited molecules tend to loosen up their intramolecular bonding, resulting in forming smaller-size clusters with increased mobility and circulation in human and animal bodies.

Several types of therapeutic devices have been developed over years for improving health of human body using far infrared (FIR) radiation, including the present inventor's previous patents (U.S. Pat. Nos. 6,363,285 and 6,516,229) and others (e.g. U.S. Pat. Nos. 6,591,142, 8,285,391, and 8,366,757.) These devices might have demonstrated success to some degree in the purposed applications, but only with limited effects.

The present inventor has engaged in far-infrared radiation research for decades, specifically in 3-20 um wavelength range. IR absorptions in said wavelength range have been scientifically identified, called “infrared signature,” in Organic Chemistry, and the present inventor is the first scientist to make the connection between theory and utilization, hoping to bring the technology to life for benefiting the society. Continuous research has bettered the present inventor's knowledge of FIR radiation and its effects on matters at molecular level. The gained knowledge has led the present inventor to developing enhanced IR-emitting ceramic composites in the desired 3-20 um wavelength band, which have been disclosed in U.S. Pat. No. 7,617,815 and U.S. patent application Ser. No. 12/657,961 for other applications by the present inventor.

During researching and making IR-emitting ceramic composites, the present inventor has come to realizing several facts regarding the properties and better use of the newly developed IR-emitting ceramic composites, which are disclosed herein as follows.

It is well known that the IR emission rate is proportional to the total surface area (A) of an IR-emitter comprising IR-emitting ceramic composite; the larger the total surface area is, the more radiation the IR-emitter may supply. However, the first fact that the present inventor has learned is that, in addition, the emission strength is also proportional to the photon generation rate of the IR-emitter, which is closely related to the total mass (M) of the IR-emitting ceramic composite. In order to have highest possible emission strength, the IR-emitter must be designed to have an optimal total-mass to total-surface-area ratio (M/A), suggesting a well-engineered IR-emitting ceramic plate would outperform a mere aggregate of IR-emitting oxide powders or particles that are disposed in forms of pads, sheets, or foams, as disclosed in prior art.

The second fact the present inventor has learned is that it would be difficult to design a broadband IR-emitter that could have uniformly distributed its radiation energy over the entire 3-20 um wavelength range. In theory, the majority of available radiation energy from an IR-emitter is often associated with shorter wavelengths (i.e. higher frequencies), governed by the energy formula for electromagnetic (EM) waves: E=hv, where h is Planck constant, and v frequency. Moreover, the peak wavelength where the maximum flux density per unit wavelength interval emerging from an IR-emitter will displace toward shorter wavelengths as the temperature of the emitter increases, known as Wien's Displacement Law. This inevitably results in radiation energy being over-strengthened in short wavelengths and meantime weakened in long wavelengths, which may leave some groups of water molecules and/or chemical compounds in human or animal body unexcited or less-excited.

To overcome this problem, the present inventor has disclosed in his previous patent (U.S. Pat. No. 7,617,815) that the desirable IR-emitters with specific peak wavelength and spectral luminance profile in 3-20 um wavelength range can be respectively made with selected oxides. In practice, the use of various IR-emitters in different wavelength bands will enhance the overall IR activation effect on molecules in body fluids and thus magnify the healing effects.

The third fact the present inventor has learned is that adding an effective amount of pyroelectric material into the mixture of IR-emitting metal oxides would significantly increase the resultant emissions at the desired 3-20 um wavelengths.

By definition, pyroelectricity is the ability of certain materials to generate temporary voltage when they are heated or cooled. The change in temperature slightly modifies the positions of the atoms in the crystal structure, such that the polarization of the material changes. This polarization change gives rise to a voltage across the crystal. If the temperature stays constant at its new value, the pyroelectric voltage will gradually disappear due to leakage current. A large ceramic plate would be less susceptible to temperature change than a smaller plate. Consequently, it is better to use an array of small IR-emitting ceramic plates for utilizing the pyroelectricity of the material than to simply use a large plate having the same volume and mass as the total of all small plates. Furthermore, a locally administrable heating device may be used to control the temperature of each IR-emitting ceramic plate for manipulating the pyroelectricity of the plates, which would dramatically increase the healing effects of the present invention on the targeted body part.

Based on aforementioned findings, the present inventor has discovered a new approach using an array of IR-emitting ceramic plates in an attachment means for enhanced IR healing effects on human or animal body, which differs from all therapeutic devices disclosed in prior art that have been implemented in forms of powders, pads, sheets, beads, or a single large plate.

### OBJECTS AND ADVANTAGES

Accordingly, one object of this invention is to provide a therapeutic device with amplified infrared emission in the desirable 3-20 um wavelength range;

Another object of the present invention is to provide a therapeutic device for effectively increasing the IR healing effects on human or animal body;

Also, another object of the present invention is to provide a simple, easy-to-use, and maintenance-free therapeutic device that is flexible to attach to any part of human or animal body that requires treatment.

These objectives are achieved by a therapeutic device comprising an array of IR-emitting elements. Said IR-emitting element is an IR-emitting ceramic plate made of essentially a mixture of selected IR-emitting metal oxides having specific spectral luminance covering at least a part of 3-20 um wavelength spectrum. Said array of IR-emitting elements can be secured in a flexible attachment means and placed at close proximity of the body part that requires treatment.

Other objects, features, and advantages of the present invention will hereinafter become apparent to those skilled in the art from the following description.

### SUMMARY OF THE INVENTION

In accordance with the present invention a therapeutic device comprises an array of IR-emitting elements in an attachment means that follows the contour of the body part to be treated, said IR-emitting element being an IR-emitting ceramic plate made of a mixture of IR-emitting oxides having specific spectral luminance covering at least a part of 3-20 um (micrometer) wavelength spectrum, providing an effective means to healing human or animal body. A locally administrable heating means may be used to provide localized heating for escalated healing effects.

### DRAWING

FIG. 1 shows a perspective view of one embodiment of the present invention with an array of two (rows) by six (columns) IR-emitting ceramic plates disposed on a substrate.

FIG. 2 shows a perspective view of another embodiment of the present invention with an array of two-by-six IR-emitting ceramic plates sandwiched by a covering sheet and a heating means in an encasement.

Reference Numerals in Drawings

| | |
|---|---|
| 11 Infrared-emitting ceramic plate | 12 Attachment means |
| 21 Heating element | 22 Encasement means |
| 31 Covering means | |

### DETAILED DESCRIPTION OF THE INVENTION

The IR-emitting ceramic plates of the present invention can be fabricated by the following process. First, prepare a mixture of powders of IR-emitting metal oxides, such as disclosed in U.S. Pat. Nos. 6,363,285, 6,516,229, and 7,617,815 by the present inventor, in theoretical percentages by weight for intended peak wavelengths and spectral luminance. An effective amount of pyroelectric material may be added to the mixture for amplified IR emissions. The pyroelectric material, tourmaline in the present invention, may be any of 5-40% by weight. Lastly, the mixture of metal oxides and pyroelectric material, along with bonding agents, catalysts, and stabilizers, is press-molded to the desired shapes and sintered in a furnace at a temperature of 1100 deg. C. or above. Several samples were prepared accordingly for concept-demonstrating experiments of the present invention.

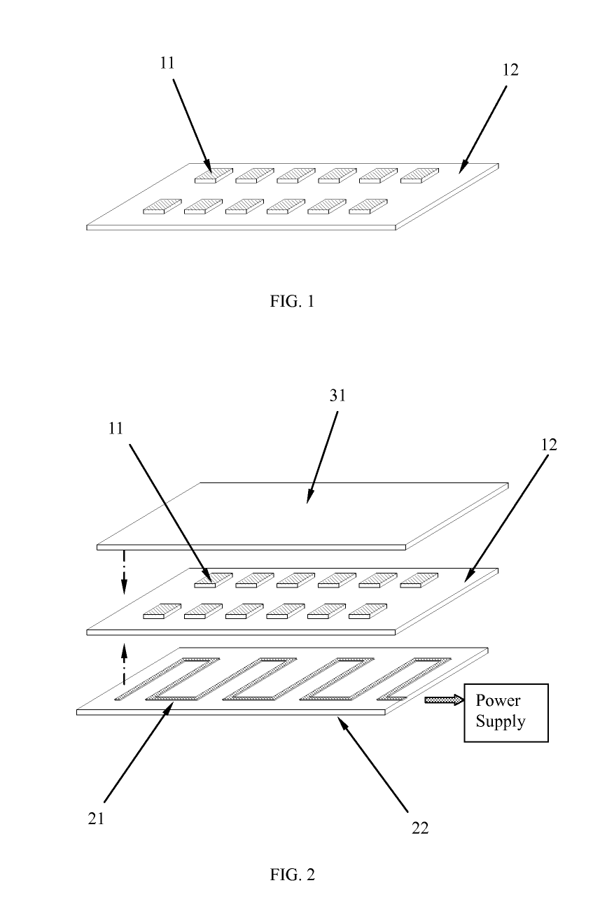

FIG. 1 shows a perspective view of an embodiment of the present invention, in which twelve IR-emitting ceramic plates 11 are mounted on a substrate 12, in a formation of two (rows) by six (columns), or a 2×6 array, which may be easily wrapped around the body part that requires treatment. In other deployments, IR-emitting ceramic plates may be secured simply by wires or strings to keep the formation. The ceramics of the present invention can take any sizes, shapes, forms, styles, patterns, and in any thickness, though a rectangular or circular plate is preferred for the ease of fabrication. Depending on the applications, the plates may have dimensions as small as in a 2-mm-diameter circle (or a 2 mm by 3 mm rectangle) up to a 50-mm-dia. circle (or 40 mm by 50 mm rectangle), with a thickness from 1 mm up to 10 mm.

FIG. 2 shows a perspective view of another embodiment of the present invention that the 2×6 array of IR-emitting ceramic plates 11 shown in FIG. 1 are sandwiched by a covering sheet 31 and a heating element 21 in an encasement 22.

The IR-emitting ceramic plates 11 absorb ambient radiation heat and convert the heat into IR photons. IR radiation from the IR-emitter 11 will last indefinitely, whenever the ambient temperature is greater than Absolute Zero, 0 deg. K or −273 deg. C. Evidently, body heat is a natural heat source for the IR-emitters. Yet, an external heat source may be used in the case that both intensified IR emission and shorter IR wavelengths are required for accelerated healing. As described before, the peak wavelengths and spectral luminance of the IR-emitters 11 will displace toward shorter wavelengths as their temperatures increase.

The dominating content of body fluids is body water. Approximately 60-65% of body water is contained within the cells (in intracellular fluid) with the other 35-40% of body water contained outside the cells (in extracellular fluid). This fluid component outside of the cells includes the fluid between the cells (interstitial fluid), lymph and blood. It is worth noting that water can absorb infrareds at wavelengths around 2.87, 3.05, 3.65, and 6.08 um, which all place at the lower end of the IR-emitter's 3-20 um wavelength spectrum. The use of an heating device will drive IR energy toward shorter wavelengths, ideal for accelerated healing that requires a strong absorption of IR emissions by body water.

Several ceramic plate samples were designed and made for concept demonstration. The base mixture of IR-emitting oxides contain, by weight, 20% silicate, 20% alumina, 24% zirconia, 4% sodium monoxide, 3% potassium oxide, 3% ferric oxide, 5% chromic oxide, 4% cobalt oxide, 2% all other minority oxides, and 15% tourmaline. Various samples with distinctive weight percentages (wt. %) of aforementioned ingredients in the base mixture were made for tailored peak wavelength and specific spectral luminance profile, specifically emphasizing on 3-7 um wavelength range for a better IR activation effect on body fluids.

Two sizes of ceramic plates were made: “Sample-A” (3 mm long, 3 mm wide, and 1 mm thick) and “Sample-B” (30 mm long, 20 mm wide, and 5 mm thick). “Sample-A” ceramic plates were used for the treatment of gum inflammation, tooth pain, and pyorrhea alveolaris, while “Sample-B” ceramic plates were used for the treatment of sprains, strains, muscular spasm, phantom limb sensation, peripheral vascular diseases, and rheumatoid arthritis. In some of the cases a heating device was required. Encouraging results have been observed.

### CONCLUSION, RAMIFICATIONS, AND SCOPE

According to the present invention, a therapeutic device comprises an array of IR-emitting elements in an attachment means, said IR-emitting element being an IR-emitting ceramic plate made of a mixture of IR-emitting oxides having specific spectral luminance covering at least a part of 3-20 (micrometer) um wavelength spectrum, for providing an effective means to healing human or animal body.

The invention has been described above. Obviously, numerous modifications and variations of the present invention are possible in light of the above teachings. Such variations are not to be regarded as a departure from the spirit and scope of the invention and all such modifications as would be obvious to one skilled in the art are intended to be included within the scope of the following claims.

I claim:

1. A therapeutic device for the treatment of human or animal body and for thereby escalating the healing effects on the ailing body, said device comprising an array of infrared-emitting elements in an attachment means disposed around the body part to be treated, said infrared-emitting element being an infrared-emitting ceramic plate made of a mixture of infrared-emitting oxides having specific spectral luminance covering at least a part of 3-20 um (micrometer) wavelength spectrum and said ceramic plate having at least a 4 square millimeter surface area for emitting said infrared toward the body.

2. A device according to claim 1 wherein said mixture of infrared-emitting oxides contain at least one of the following oxides: silicate, alumina, zirconia, sodium monoxide, potassium oxide, ferric oxide, chromic oxide, cobalt oxide, magnesium oxide, lithium oxide, calcium oxide, and titanium oxide.

3. A device according to claim 1 wherein 5-40 wt. % (weight percentage) of said mixture of infrared-emitting oxides is replaced with pyroelectric material, comprising tourmaline.

4. A device according to claim 1 wherein said infrared-emitting element is heated by a heating means for escalated healing effects.

5. A method for a noninvasive treatment of human or animal body for escalated healing effects, said method comprising:
a) providing a human or animal body;
b) providing an attachment means placed around said body;
c) providing an array of infrared-emitting elements disposed in said attachment means, said infrared-emitting element being an infrared-emitting ceramic plate made of a mixture of infrared-emitting oxides having specific spectral luminance covering at least a part of 3-20 um (micrometer) wavelength spectrum and said ceramic plate having at least a 4 square millimeter surface area for emitting said infrared toward said body; and
d) providing an optional heating means for increasing the temperature of said infrared-emitting elements to achieve escalated healing effects.
